(19) United States
(12) Patent Application Publication
(10) Pub. No.: US 2026/0234083 A1
(43) Pub. Date: Aug. 13, 2026

(54) SYSTEM AND METHOD FOR INTEGRATED LIVESTOCK METHANE CAPTURE, PROCESSING, AND CONVERSION TO HIGH-VALUE PRODUCTS

(71) Applicant: Planet.Tech, Austin, TX (US)

(72) Inventor: Griffin C. Cherry, Austin, TX (US)

(21) Appl. No.: 19/537,006

(22) Filed: Feb. 11, 2026

Related U.S. Application Data
(60) Provisional application No. 63/758,073, filed on Feb. 13, 2025.

(51) Int. Cl. C07C 7/144; A01K 29/00; B01D 53/22; C07C 29/48

(52) U.S. Cl. CPC C07C 7/144; A01K 29/00; B01D 53/229; C07C 29/48; A01K 2227/101; B01D 2257/7025

(57) ABSTRACT

A system and method for capturing, processing, and converting livestock methane emissions into valuable hydrocarbon products. The disclosure includes a wearable methane collection apparatus connected to a centralized processing network, integrating smart pressure regulation, automated transfer mechanisms, and AI-optimized processing capabilities. This system enables both environmental protection and value generation through the conversion of waste methane into high-value products.

### CROSS-REFERENCE TO RELATED APPLICATION

This application claims the benefit of and priority to U.S. Provisional Patent Application No. 63/758,073 filed on Feb. 13, 2025, the entire disclosure of which is hereby incorporated by reference in its entirety for all purposes.

### TECHNICAL FIELD

The application relates generally to environmental technology, renewable energy production, and sustainable agriculture, and more specifically to systems and methods for capturing, processing, and converting livestock methane emissions into valuable hydrocarbon products through a distributed collection and centralized processing network.

### BACKGROUND OF THE DISCLOSURE

Livestock methane emissions represent a significant environmental challenge due to methane's high global warming potential relative to carbon dioxide. A substantial portion of these emissions arises from enteric fermentation in ruminant animals and is released directly into the atmosphere during normal biological processes such as belching. As livestock populations increase globally, the cumulative impact of these emissions has become an area of growing regulatory, environmental, and economic concern. Despite this impact, methane emitted from livestock remains largely unmanaged at the point of release.

Existing approaches to addressing livestock methane emissions primarily focus on reducing methane generation rather than capturing and utilizing the emitted gas. Such approaches include dietary interventions, feed additives, breeding strategies, and herd management practices. While these methods may reduce emissions to some extent, they do not recover methane for productive use and therefore forgo its potential value as an energy or chemical feedstock. Other solutions rely on centralized or facility-scale infrastructure, such as anaerobic digestion systems, which are typically limited to manure management and require significant capital investment, fixed installations, and operational complexity. These infrastructure-intensive solutions are often difficult to scale across distributed livestock operations and do not directly address methane released during enteric emissions, leaving a substantial portion of emissions unaddressed.

### SUMMARY OF THE DISCLOSURE

The present disclosure provides a comprehensive system and method for capturing, processing, and converting livestock methane emissions into valuable products. The disclosure is configured to capture methane at or near the point of emission, safely manage and transport the captured gas, and convert the methane into market-ready outputs, thereby integrating environmental mitigation with economic value generation.

In one embodiment, the disclosure includes a wearable methane collection apparatus configured to be worn by a livestock animal. The collection apparatus includes intelligent pressure regulation and adaptive sealing features that enable efficient methane capture while maintaining animal comfort and mobility.

The disclosure further includes distributed storage and transfer mechanisms configured to temporarily store captured methane and transport the methane from distributed collection locations to one or more centralized processing facilities. The distributed architecture enables scalable deployment across livestock operations of varying size and geography.

A centralized processing hub architecture is provided for purification, conditioning, and conversion of captured methane. The centralized hub may include gas separation, compression, bulk storage, and conversion systems configured to prepare methane for downstream utilization.

Automated collection and monitoring systems are integrated throughout the disclosure to enable real-time sensing, data collection, and operational control. These systems support continuous operation, reduce manual intervention, and improve capture efficiency and safety.

The disclosure further includes a network optimization and control infrastructure configured to manage system-wide functionality. The control infrastructure coordinates distributed and centralized components, applies analytics and optimization algorithms, and supports predictive maintenance, safety enforcement, and regulatory compliance.

Finally, the disclosure includes value conversion and distribution methods configured to convert processed methane into hydrocarbons or other valuable products and to prepare such products for commercial distribution. Through this integrated approach, the disclosure transforms livestock methane emissions from an environmental liability into a scalable and monetizable resource.

The foregoing is a summary and thus contains, by necessity, simplifications, generalizations and omissions of detail; consequently, the summary is illustrative only and is not limiting in any way. Other aspects, inventive features, and advantages of the systems and/or processes described herein may become apparent in the non-limiting detailed description set forth herein.

### BRIEF DESCRIPTION OF THE DRAWINGS

These and other features, aspects, and advantages of the present disclosure may become better understood with regard to the following description, and accompanying drawings, where:

Figure (FIG. 1 illustrates a livestock-mounted methane capture and storage system in accordance with one embodiment of the present disclosure.

FIG. 2 illustrates a piping and instrumentation diagram (P&ID) for an integrated livestock methane capture, processing, and storage system in accordance with one embodiment of the disclosure.

### DETAILED DESCRIPTION

To make the objectives, features and advantages of the present disclosure more obvious and understandable, the present disclosure will be further described hereinafter with reference to the accompanying drawings and specific embodiments.

It should be noted that specific details are set forth in the following description to facilitate understanding the present disclosure. However, the present disclosure may be implemented in many other ways different from those described herein, and those skilled in the art may make similar generalizations without violating the connotation of the present disclosure. Therefore, the present disclosure is not limited by the specific embodiments disclosed below.

The terms used in the embodiments of the present disclosure are merely for the purpose of describing specific embodiments, and are not intended to limit the present disclosure. The singular forms of “a”, “said” and “the” used in the embodiments of the present disclosure and the appended claims are also intended to include plural forms, unless the context clearly indicates other meanings.

It should be noted that the example embodiments may be implemented in various forms, and should not be construed as being limited to the embodiments set forth herein. On the contrary, the provision of these embodiments makes the present disclosure more comprehensive and complete, and fully conveys the concept of the example embodiments to those skilled in the art. The same reference numerals in the figures indicate the same or similar structures, and thus their repeated description may be omitted. In addition, the similarities between the embodiments may not be repeated.

### Collection System

### Wearable Device Architecture

### A. Capture Mechanism

In one embodiment, the collection system includes a wearable methane capture device configured to be worn by a livestock animal, such as a ruminant, during normal activity. The capture mechanism is designed to collect methane-rich gases expelled during enteric emission events while minimizing interference with the animal's comfort, behavior, and mobility.

The capture mechanism includes a flexible membrane constructed from compliant polymeric, elastomeric, or composite materials. The flexible membrane conforms to the contours of the animal's muzzle or mouth region, allowing effective gas capture while accommodating natural movement. The material selection and structural design provide durability, chemical resistance, and low gas permeability, while maintaining softness and flexibility to enhance comfort and long-term wearability.

Smart pressure regulation is integrated into the capture mechanism to optimize methane collection efficiency. The system maintains a controlled pressure differential sufficient to draw expelled gases into the collection pathway without creating excessive suction or resistance that could cause discomfort or disrupt normal breathing. Pressure regulation may be dynamically adjusted based on sensor inputs, including detected emission events and gas flow conditions.

An adaptive sealing system is provided to reduce gas leakage during operation. The sealing system may include compliant edges, adjustable interfaces, or self-adjusting sealing elements that accommodate variations in animal anatomy and movement. This adaptive sealing helps ensure consistent gas capture while avoiding rigid or constrictive contact with the animal.

Weight reduction techniques are incorporated throughout the wearable device to minimize added load on the animal. Lightweight materials, compact component integration, and distributed mass placement are employed to reduce strain and preserve natural mobility. The overall design prioritizes balance and stability to prevent shifting or chafing during movement.

Animal welfare and ergonomic considerations are integral to the design of the capture mechanism. The device is configured to allow normal feeding, drinking, and respiration, and to avoid sharp edges, pressure points, or obstructive elements. The wearable architecture is intended to support extended use without causing stress, injury, or behavioral disruption, thereby enabling continuous and reliable methane capture in real-world agricultural environments.

### B. Storage Component

In one embodiment, the wearable device architecture further includes one or more storage components configured to temporarily store captured methane gas prior to downstream processing, separation, or transfer. The storage components are designed to safely contain methane under controlled conditions while maintaining compatibility with wearable and modular deployment.

The storage components may incorporate compression mechanisms configured to increase the density of captured methane and reduce storage volume. Such compression mechanisms may include mechanical compressors, diaphragm-based systems, or pressure-assisted flow arrangements driven by vacuum or pump systems located upstream or downstream of the collection device. Compression levels are selected to balance storage efficiency with safety and energy consumption.

To prevent overpressure conditions, the storage components include one or more safety release systems. These systems may include pressure relief valves, burst disks, venting pathways, or automated shutoff controls that activate when internal pressure exceeds a predetermined threshold. Safety release systems are configured to protect both the animal and the equipment while ensuring controlled and predictable pressure dissipation.

Volume optimization features are incorporated to maximize storage efficiency within the physical constraints of a wearable or modular system. Such features may include expandable chambers, segmented storage volumes, or flexible containment structures that adapt to varying gas quantities. The storage geometry may be configured to distribute mass evenly and minimize bulk while maintaining structural integrity.

Pressure monitoring is provided through one or more sensors configured to continuously or intermittently measure internal storage pressure. Pressure data may be used by a control system to regulate compression, initiate safety responses, or control downstream transfer of methane. Monitoring ensures stable operation and enables automated feedback control to maintain pressure within safe operating limits.

The storage components are constructed from durable, lightweight materials selected to withstand repeated pressure cycles, environmental exposure, and mechanical stress associated with animal movement. Materials may include reinforced polymers, composite laminates, or coated metals that provide strength, corrosion resistance, and reduced weight. The resulting structure supports long-term use in agricultural environments while minimizing impact on animal comfort and mobility.

### C. Control System

In one embodiment, the collection system further includes an integrated control system configured to monitor, regulate, and optimize operation of the methane capture and storage components. The control system coordinates sensing, data processing, and actuation functions to enable automated, reliable, and efficient system performance.

The control system includes IoT-enabled sensors integrated throughout the collection, storage, and gas handling subsystems. These sensors may include, but are not limited to, acoustic sensors, accelerometers, gas concentration sensors, pressure sensors, flow sensors, and oxygen analyzers. Sensor data is collected in real time to monitor emission events, gas composition, flow conditions, and storage status.

Automated data collection and analysis are performed by a local controller, remote computing resource, or a combination thereof. Sensor data may be processed continuously or intermittently to detect methane emission events, adjust operating parameters, and log performance metrics. Data aggregation enables historical analysis, system diagnostics, and compliance reporting.

Performance optimization may be achieved through AI-based analytics or algorithmic control models. Such analytics may be used to identify emission patterns, optimize vacuum timing, regulate gas flow rates, and improve separation efficiency. Machine learning models may adapt system behavior over time based on observed animal behavior, environmental conditions, or operational constraints, thereby improving capture efficiency while minimizing energy consumption and animal impact.

Built-in safety protocols are incorporated to ensure system reliability and safe operation. These protocols may include threshold-based alarms, automatic shutdown or isolation of system components, pressure and flow limit enforcement, and fault detection routines. Safety logic may be executed locally to ensure rapid response independent of network connectivity.

Wireless communication capabilities are provided to enable remote monitoring and management of the system. Communication may be implemented using Wi-Fi, cellular, low-power wide-area networks, Bluetooth, or other wireless protocols. Remote connectivity allows system status reporting, parameter updates, firmware upgrades, and centralized management across multiple deployed units, facilitating scalable operation in distributed agricultural environments.

### Processing Infrastructure

### Centralized Hub Design

### A. Collection Network

In one embodiment, the processing infrastructure includes a centralized hub configured to receive methane collected from a plurality of distributed livestock-mounted capture systems. The centralized hub functions as an aggregation, processing, and control point, enabling scalable management of methane captured across multiple animals or facilities.

The collection network includes automated transfer systems configured to transport methane from individual storage components or intermediate collection points to the centralized hub. Transfer may be accomplished using pressurized pipelines, flexible conduits, mobile transfer units, or removable storage modules. Automated valves, pumps, or compressors may be employed to regulate flow and ensure consistent delivery without manual intervention.

Route optimization is implemented to enhance transfer efficiency and reduce energy consumption. The system may dynamically select transfer pathways, timing, or sequencing based on factors such as methane volume, pressure levels, distance to the hub, and network availability. Optimization algorithms may be executed by a central control system to coordinate transfer operations across multiple sources.

Continuous quality monitoring is provided throughout the collection network to ensure consistency of the methane stream delivered to the centralized hub. Sensors may be deployed at transfer interfaces or along transport routes to measure methane concentration, oxygen content, moisture levels, and other quality parameters. Data from these sensors may be used to validate gas purity, identify anomalies, or adjust processing parameters.

Safety protocols are integrated to ensure secure handling of methane during transport. Such protocols may include leak detection, pressure relief mechanisms, automatic shutoff valves, and fault isolation routines. In the event of abnormal conditions, the system may initiate controlled venting, flow interruption, or network reconfiguration to maintain safe operation.

Integrated distribution mechanisms are provided to facilitate downstream processing, storage, or utilization of methane received at the centralized hub. These mechanisms may include manifold systems, buffering tanks, metering devices, or interfaces to conversion equipment. The collection network thereby supports seamless transition from distributed capture to centralized processing within a unified infrastructure.

### B. Processing Systems

In one embodiment, the centralized hub further includes one or more processing systems configured to condition, purify, compress, and prepare captured methane for storage, transport, or downstream utilization. The processing systems are designed to operate on methane received from multiple distributed collection sources while maintaining consistent quality and operational efficiency.

Advanced purification techniques are employed to remove impurities from the methane stream. Such impurities may include oxygen, nitrogen, carbon dioxide, moisture, particulate matter, or trace contaminants. Purification may be accomplished using membrane separation, pressure swing adsorption, adsorption media, catalytic treatment, filtration, or combinations thereof. The selection and sequencing of purification stages may be adjusted based on measured gas composition and target end-use requirements.

Compression technology is utilized to increase methane density for energy-efficient storage and transport. Compression systems may include mechanical compressors, multi-stage compression units, or electrically driven compression devices configured to minimize energy consumption while achieving desired pressure levels. Compression parameters may be dynamically controlled in response to storage capacity, downstream demand, or operational constraints.

Scalable storage solutions are provided to accommodate bulk quantities of processed methane. Storage systems may include pressurized tanks, modular storage vessels, or expandable containment systems designed for incremental capacity expansion. The storage architecture enables the centralized hub to scale with increased methane capture volumes without requiring substantial redesign or interruption of operation.

Rigorous quality control measures are implemented throughout the processing systems to ensure methane meets predetermined purity, pressure, and safety specifications. Sensors and analytical instruments may continuously or periodically measure gas composition, moisture content, pressure, and temperature. Quality data may be logged, analyzed, and used to trigger corrective actions or process adjustments as needed.

Secure distribution preparation systems are included to enable delivery of processed methane to downstream markets or utilization pathways. These systems may include metering devices, pressure regulation interfaces, safety isolation valves, and standardized transfer connections. The processed methane may thereby be prepared for use in energy generation, chemical conversion, transportation fuel, or other commercial applications while maintaining compliance with applicable safety and regulatory standards.

### C. Control Infrastructure

In one embodiment, the centralized hub includes a control infrastructure configured to provide real-time oversight, coordination, and optimization of the collection network and processing systems. The control infrastructure functions as an integrated management layer that aggregates data from distributed capture devices, transfer systems, and processing equipment to enable centralized monitoring and control.

Network management capabilities are provided to support real-time oversight of system operation. The control infrastructure monitors status, performance, and availability of collection sources, transport pathways, and processing components. Based on this information, the system may coordinate gas transfer scheduling, balance loads across processing units, and respond dynamically to changing operating conditions.

Predictive performance analytics are employed to anticipate system behavior and optimize operational efficiency. Historical and real-time data may be analyzed to forecast methane production rates, identify equipment wear or degradation, and predict maintenance needs. Predictive analytics enable proactive system adjustments that reduce downtime and improve reliability.

Regulatory compliance tracking is integrated within the control infrastructure to support adherence to applicable environmental, safety, and reporting requirements. The system may log methane capture volumes, emissions reduction metrics, gas quality data, and operational events. Such records may be used for compliance verification, audit support, incentive programs, or carbon credit accounting.

AI-driven optimization techniques may be applied to improve system efficiency and performance. Machine learning models or algorithmic optimization routines may analyze patterns across the network to optimize collection timing, processing parameters, storage utilization, and energy consumption. These models may continuously adapt based on operational feedback, enabling system-wide performance improvements over time.

System-wide data integration and monitoring are achieved through centralized data aggregation and visualization. Data from sensors, controllers, and subsystems are integrated into a unified platform that supports real-time dashboards, alerts, and remote management interfaces. This integrated monitoring enables coordinated control across the entire methane capture, processing, and distribution infrastructure while supporting scalable deployment across multiple sites.

FIG. 1 illustrates a livestock-mounted methane capture and storage system 100 in accordance with one embodiment of the present disclosure. The figure depicts a ruminant animal 102 wearing a wearable methane collection device 104 positioned at or near the animal's mouth and muzzle region. The collection device 104 is integrated with a halter or harness and is configured to capture methane-rich gases emitted during enteric fermentation events. Captured gases are conveyed through a flexible conduit extending from the collection device 104 to a compact processing and storage assembly 106 mounted on or adjacent to the animal.

The processing and storage assembly 106 includes a vacuum pump or fan 108 configured to generate negative pressure for drawing gases from the collection device 104. The assembly further includes power and communication components 110, control electronics and sensor modules 112 configured to monitor system operation and gas characteristics. An oxygen separation module 114 is positioned downstream of the collection pathway and is configured to remove oxygen and other non-methane gases from the captured gas stream.

Methane-rich gas exiting the oxygen separation module is directed into a methane storage container 116 configured to safely store captured methane. The storage container 116 is shown as a pressurized vessel labeled for methane storage and is sized and positioned to minimize impact on animal mobility and comfort. Control and sensing components 112 are integrated within the assembly 106 to enable automated operation, safety monitoring, and data communication.

FIG. 1 illustrates the overall system architecture and physical integration of the wearable methane capture device, gas conveyance pathway, processing components, oxygen separation module, and methane storage subsystem as deployed on a livestock animal. The configuration shown is exemplary and intended to demonstrate functional relationships among system components rather than to limit the disclosure to a specific form factor or mounting arrangement.

### Network Architecture

### System Integration

### A. Data Management

In one embodiment, the network architecture includes a data management subsystem configured to integrate, process, and analyze data generated across the methane capture, storage, transfer, and processing infrastructure. The data management subsystem enables coordinated operation, performance optimization, and compliance tracking across distributed and centralized system components.

Real-time monitoring is provided to track methane capture events, gas flow, storage conditions, and processing status. Data collected from sensors associated with wearable collection devices, storage components, transport systems, and centralized processing equipment are transmitted to one or more data processing nodes. This real-time visibility enables prompt detection of operational changes and supports responsive control actions.

Performance analytics are applied to system data to optimize overall system output. Analytical models may evaluate capture efficiency, transfer utilization, processing throughput, and storage capacity to identify opportunities for improvement. Insights derived from performance analytics may be used to adjust control parameters, resource allocation, or operational scheduling to enhance efficiency and maximize methane recovery.

Predictive maintenance functionality is incorporated to improve system longevity and reliability. By analyzing trends in sensor readings, operating conditions, and historical performance data, the system may identify early indicators of component wear, fouling, or failure. Maintenance actions may be scheduled proactively, reducing unplanned downtime and extending the useful life of system components.

Quality control and regulatory compliance tracking are integrated into the data management subsystem. Gas composition, pressure, temperature, and operational events may be logged and retained in accordance with applicable standards and reporting requirements. This data supports verification of methane purity, validation of emissions reduction, and documentation for regulatory, safety, or incentive-based programs, including environmental reporting and carbon accounting.

### B. Communication Systems

In one embodiment, the network architecture includes a communication system configured to enable secure data exchange and control signaling among distributed methane capture devices, storage components, transport systems, and centralized processing infrastructure. The communication system supports reliable connectivity across geographically distributed assets while maintaining data integrity and operational security.

Network management capabilities are provided to support real-time oversight of system operation. The control infrastructure monitors status, performance, and availability of collection sources, transport pathways, and processing components. Based on this information, the system may coordinate gas transfer scheduling, balance loads across processing units, and respond dynamically to changing operating conditions.

Predictive performance analytics are employed to anticipate system behavior and optimize operational efficiency. Historical and real-time data may be analyzed to forecast methane production rates, identify equipment wear or degradation, and predict maintenance needs. Predictive analytics enable proactive system adjustments that reduce downtime and improve reliability.

Regulatory compliance tracking is integrated within the control infrastructure to support adherence to applicable environmental, safety, and reporting requirements. The system may log methane capture volumes, emissions reduction metrics, gas quality data, and operational events. Such records may be used for compliance verification, audit support, incentive programs, or carbon credit accounting.

AI-driven optimization techniques may be applied to improve system efficiency and performance. Machine learning models or algorithmic optimization routines may analyze patterns across the network to optimize collection timing, processing parameters, storage utilization, and energy consumption. These models may continuously adapt based on operational feedback, enabling system-wide performance improvements over time.

System-wide data integration and monitoring are achieved through centralized data aggregation and visualization. Data from sensors, controllers, and subsystems are integrated into a unified platform that supports real-time dashboards, alerts, and remote management interfaces. This integrated monitoring enables coordinated control across the entire methane capture, processing, and distribution infrastructure while supporting scalable deployment across multiple sites.

### C. Optimization Methods

In one embodiment, the network architecture includes one or more optimization methods configured to improve efficiency, safety, and cost-effectiveness of methane capture, transport, processing, and storage across the integrated system. The optimization methods operate using data collected from distributed sensors, control systems, and processing infrastructure.

AI-based route planning is employed to optimize methane transport within the collection network. Machine learning models or algorithmic optimization routines may evaluate variables including methane volume, pressure levels, transport capacity, distance, energy consumption, and network availability to determine optimal transfer routes and timing. Route planning may be dynamically updated in response to changing operational conditions, enabling efficient aggregation of methane from multiple sources to centralized processing hubs.

Resource allocation methods are used to balance operational cost and system efficiency. The system may dynamically allocate compression capacity, storage availability, transport resources, and processing throughput based on real-time demand and predicted methane generation. Optimization routines may seek to minimize energy usage, reduce idle capacity, and prioritize high-value processing pathways while maintaining system performance.

Safety and quality control assurance are integrated into the optimization methods to ensure reliable and compliant operation. Optimization algorithms may incorporate safety constraints, pressure limits, purity thresholds, and regulatory requirements as hard boundaries within decision-making processes. If unsafe or non-compliant conditions are detected or predicted, the system may override optimization outputs to initiate protective actions, adjust operating parameters, or isolate affected components.

These optimization methods enable coordinated, adaptive, and intelligent control of the methane capture and processing network, supporting scalable deployment while maintaining safety, quality, and economic viability.

### Value Creation Methods

### Processing and Conversion

### A. Methane Processing

In one embodiment, the system includes methane processing methods configured to convert captured livestock methane into a form suitable for storage, transport, and commercial utilization. Methane processing is performed after collection and aggregation and is designed to enhance gas quality, ensure safe handling, and enable integration with downstream markets.

Purification techniques are applied to refine captured methane by removing residual impurities that may remain after initial separation. Such impurities may include oxygen, nitrogen, carbon dioxide, moisture, or trace contaminants. Purification may be accomplished using membrane separation, adsorption media, pressure swing adsorption, filtration, catalytic treatment, or combinations thereof. The purification process may be dynamically adjusted based on measured gas composition and target specifications for intended end use.

Storage solutions for processed methane are provided to safely contain methane following purification. Storage systems may include pressurized tanks, modular storage vessels, or bulk containment units designed to accommodate varying volumes. Storage parameters such as pressure and temperature may be actively monitored and controlled to maintain stability and prevent degradation of gas quality.

Safety and efficiency protocols are incorporated throughout the methane processing workflow. These protocols may include pressure relief systems, leak detection, automated shutdown mechanisms, and controlled venting pathways. Operational efficiency may be enhanced through optimized process sequencing, energy management, and automated control logic that minimizes losses while maintaining safe operating conditions.

Market distribution readiness is achieved by conditioning processed methane to meet applicable quality, pressure, and regulatory requirements for downstream use. The processed methane may be prepared for delivery to energy markets, chemical feedstock applications, transportation fuel systems, or other commercial pathways. Metering, certification, and transfer interfaces may be provided to facilitate integration with existing distribution infrastructure and market mechanisms.

### B. Product Conversion

In one embodiment, the system includes product conversion methods configured to transform processed methane into one or more high-value products. Product conversion enables utilization of captured methane as a feedstock for fuels, chemicals, or other hydrocarbons, thereby converting waste emissions into commercially valuable outputs.

Hydrocarbon production is achieved through methane conversion processes that may include catalytic reforming, partial oxidation, steam methane reforming, autothermal reforming, Fischer-Tropsch synthesis, methanol synthesis, or other chemical conversion techniques. The selection of conversion pathways may depend on desired end products, processing scale, energy availability, and market demand. Conversion systems may be configured for centralized or modular deployment and may operate continuously or in batch modes.

Quality control measures are implemented to ensure converted products meet applicable industry standards and specifications. Product quality may be monitored using analytical instrumentation to verify composition, purity, and physical properties. Quality data may be used to adjust conversion parameters, validate compliance with regulatory or contractual requirements, and certify products for commercial distribution.

Process optimization is applied to support scalability and economic efficiency of product conversion operations. Optimization techniques may include control of reaction conditions, catalyst management, energy integration, and throughput balancing. Data-driven optimization models may adapt process parameters in response to feedstock variability, operational constraints, or market conditions, enabling efficient scaling from pilot-scale systems to commercial deployment.

Distribution infrastructure is provided to enable commercial integration of converted products. Such infrastructure may include storage vessels, metering systems, packaging or loading interfaces, and transportation connections. The converted products may be delivered to downstream users, distribution networks, or end markets in a form compatible with existing supply chains, thereby facilitating widespread adoption and monetization of captured methane.

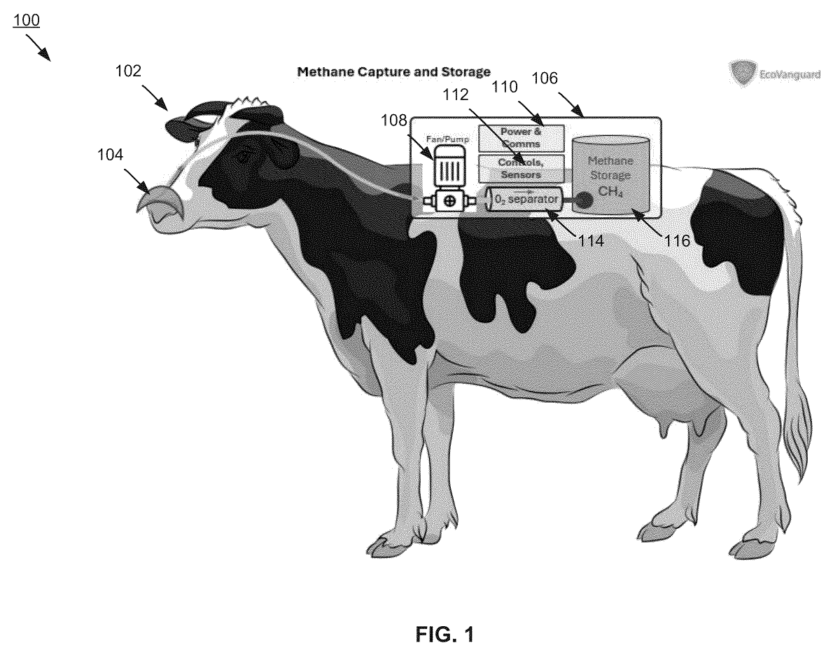

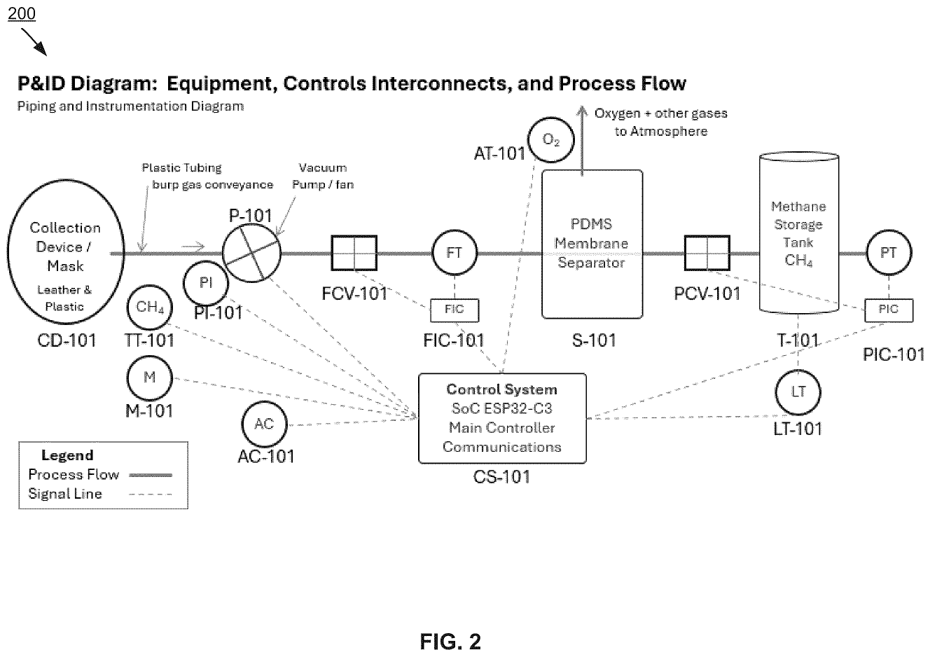

FIG. 2 illustrates a piping and instrumentation diagram (P&ID) 200 for an integrated livestock methane capture, processing, and storage system in accordance with one embodiment of the disclosure.

The diagram depicts process flow, equipment, sensing elements, control devices, and interconnections beginning at a livestock-mounted collection device (CD-101) configured to capture methane-rich gases emitted by the animal. Captured gases are conveyed through plastic tubing to a vacuum pump (P-101), which creates negative pressure to draw gases from the collection device. A pressure indicator (PI-101) monitors vacuum pressure, while a flow control valve (FCV-101) and flow transmitter/controller (FIC-101) regulate and monitor gas flow through the system.

Upstream sensing elements include a methane sensor (TT-101) positioned at the collection point to monitor methane concentration, a microphone or acoustic sensor (M-101) configured to detect belching events, and an accelerometer (AC-101) configured to detect vibration patterns associated with gas expulsion. These sensors provide input signals to a central control system (CS-101).

The gas stream is directed to an oxygen separation module (S-101), shown as a PDMS membrane separator, which selectively removes oxygen and other non-methane gases from the stream. Oxygen-rich exhaust is vented upward to the atmosphere, and an oxygen analyzer (AT-101) monitors oxygen concentration in the vented stream to verify separation performance.

Methane-rich gas exiting the separator is directed through a pressure control valve (PCV-101) into a methane storage tank (T-101). The storage subsystem includes a check valve (CV-101) to prevent backflow, a pressure transmitter/controller (PIC-101) to maintain safe storage pressure, and a level transmitter (LT-101) to monitor methane quantity within the tank.

Dashed lines in the diagram represent signal and control connections between sensors, controllers, and actuated components. The central control system (CS-101), implemented in one embodiment using an ESP32-C3 system-on-chip or similar controller, receives input from sensing devices and executes automated control loops for vacuum regulation, flow control, oxygen separation verification, and storage pressure management.

The accompanying component reference table below lists component tag numbers, names, and descriptions corresponding to the elements shown in the P&ID, providing a structured mapping between system hardware, sensing elements, and control functions.

| | Component | |
|---|---|---|
| # | Tag #s | Item Description |
| 1 | CD-101 | Collection Device (Left Side) The leather and plastic mask |
| | | that fits on the cow, integrated with the halter. Connected to |
| | | plastic tubing for gas conveyance |
| 2 | | Sensing and Flow Control |
| | M-101 | Microphone or acoustic sensor (M-101) detects belch event |
| | AC-101 | Accelerometer (AC-101) to detect vibration patterns from |
| | | belching |
| | TT-101 | Methane sensor (TT-101) monitors gas content at collection |
| | | point, MOS calibrated for 30% methane concentration, NDIR |
| | P-101 | Vacuum Pump (P-101) creates negative pressure, pulling gases |
| | | from the collection device |
| | PI-101 | Pressure Indicator (PI-101) monitors vacuum pressure |
| | FCV-101 | Flow control valve (FCV-101) regulates the gas flow rate |
| | FIC-101 | Flow transmitter and controller (FIC-101) provide feedback |
| | | control |
| 3 | | Oxygen Separation |
| | S-101 | PDMS membrane separator (S-101) removes oxygen from the |
| | | gas stream |
| | | Oxygen is directed upward and released to atmosphere |
| | AT-101 | Oxygen analyzer (AT-101) monitors oxygen content in the |
| | | vented stream |
| 4 | | Methane Storage |
| | PCV-101 | Pressure control valve (PCV-101) regulates flow into storage |
| | | tank |
| | T-101 | Methane storage tank (T-101) with pressure and level |
| | | monitoring |
| | CV-101 | Check Valve (CV-101) prevents backflow |
| | PIC-101 | Pressure transmitter and controller (PIC-101) maintain safe |
| | | pressure levels |
| | LT-101 | Level transmitter (LT-101) monitors methane quantity in |
| | | storage |
| 5 | | Control System |
| | CS-101 | Central controller (CS-101) receives input from all sensors, |
| | | ESP32-C3 SoC |
| | | Control signals (dashed lines) connect sensors to controllers |
| | | and valves |
| | | Automated control loops for flow and pressure management |

The foregoing description of the disclosure has been presented for purposes of illustration and description. It is not intended to be exhaustive or to limit the disclosure to the precise forms disclosed. Modifications and variations are possible in light of the above teachings or may be derived from practice of the disclosure. The embodiments described were chosen and described to best explain the principles of the disclosure and its practical application, and to enable others skilled in the art to understand the disclosure for various embodiments and with various modifications as are suited to the particular use contemplated.

Unless otherwise indicated, all terms used herein are intended to be given their ordinary and customary meaning as understood by a person of ordinary skill in the relevant art at the time of the disclosure. The use of singular terms includes the plural unless clearly indicated otherwise. The use of “or” is intended to be inclusive unless explicitly stated otherwise.

Any reference to specific materials, components, processes, or parameters is not intended to limit the disclosure unless expressly recited in the claims. Alternative materials, components, processes, and parameter ranges may be used without departing from the scope of the disclosure.

Functional blocks, components, or systems described herein may be implemented using hardware, software, firmware, or combinations thereof. Control logic, analytics, optimization routines, and decision-making processes may be implemented using programmable processors, microcontrollers, cloud-based computing resources, or distributed computing architectures, and may be embodied as non-transitory computer-readable media storing executable instructions.

The disclosure may be embodied in other specific forms without departing from the spirit or essential characteristics thereof. The scope of the disclosure is therefore indicated by the appended claims rather than by the foregoing description, and all changes which come within the meaning and range of equivalency of the claims are intended to be embraced therein.

No element, act, or instruction used in the description of the present disclosure should be construed as critical or essential unless explicitly described as such. Headings are provided for organizational convenience only and do not limit or define the scope of the disclosure.

What is claimed is:

1. A system for capturing and converting livestock methane emissions, comprising:
a wearable methane collection device configured to be worn by a livestock animal;
one or more sensors configured to detect methane emission events;
a gas conveyance pathway coupled to the collection device;
a gas processing infrastructure configured to purify and store methane; and
a control system configured to coordinate methane collection, processing, and storage.

2. The system of claim 1, wherein the wearable methane collection device comprises a flexible membrane configured to conform to a muzzle or mouth region of the livestock animal.

3. The system of claim 1, wherein the one or more sensors include at least one of an acoustic sensor, an accelerometer, a methane concentration sensor, or a pressure sensor.

4. The system of claim 1, further comprising a vacuum generation system configured to apply negative pressure during detected methane emission events.

5. The system of claim 1, wherein the wearable methane collection device includes an adaptive sealing system configured to reduce gas leakage during operation.

6. The system of claim 1, further comprising a storage component including a compression mechanism configured to increase methane storage density.

7. The system of claim 6, wherein the storage component includes a pressure relief system configured to prevent overpressure conditions.

8. The system of claim 1, wherein the gas processing infrastructure includes a gas separation module configured to separate methane from oxygen and other gases.

9. The system of claim 8, wherein the gas separation module comprises a membrane-based separation system.

10. The system of claim 1, wherein the control system includes IoT-enabled communication for real-time monitoring of system operation.

11. The system of claim 10, wherein the control system is configured to transmit operational data wirelessly to a remote management platform.

12. The system of claim 1, wherein the control system applies AI-based analytics to optimize methane capture efficiency or energy consumption.

13. A centralized methane processing system, comprising:
a collection network configured to receive methane from a plurality of distributed livestock-mounted methane capture systems;
one or more purification systems configured to refine methane quality;
one or more bulk storage systems for processed methane; and
a centralized control infrastructure configured to manage transport, processing, and storage operations.

14. The centralized methane processing system of claim 13, wherein the collection network includes automated transfer pathways selected using route optimization algorithms.

15. The centralized methane processing system of claim 13, wherein the centralized control infrastructure is configured to perform regulatory compliance tracking or emissions accounting.

16. A method for capturing and converting livestock methane emissions, comprising:
detecting a methane emission event from a livestock animal;
capturing methane-rich gas using a wearable collection device;
transporting the captured gas to a processing system;
purifying the methane; and
storing the purified methane for downstream utilization.

17. The method of claim 16, further comprising separating methane from oxygen using a membrane-based separation process.

18. The method of claim 16, further comprising applying predictive analytics to schedule maintenance or adjust operating parameters.

19. The method of claim 16, further comprising converting the purified methane into one or more hydrocarbon products.

20. The method of claim 19, wherein converting the purified methane includes catalytic reforming, methanol synthesis, or Fischer-Tropsch synthesis.
